(19) United States
(12) Patent Application Publication
(10) Pub. No.: US 2026/0053825 A1
(43) Pub. Date: Feb. 26, 2026

(54) CONSUMER PACKAGE FOR BITE TREATMENT CONTAINING A TOPICAL CREAM THAT INCLUDES A TETRACYCLINE OR THIRD GENERATION CEPHALOSPORIN

(71) Applicant: KAZM PHARMACEUTICALS INC., Hamilton (CA)

(72) Inventors: Patrick Musitano, Hamilton (CA); Paul Zalzal, Hamilton (CA)

(73) Assignee: KAZM PHARMACEUTICALS INC., Hamilton, ON (CA)

(21) Appl. No.: 19/309,757

(22) Filed: Aug. 26, 2025

Related U.S. Application Data
(63) Continuation-in-part of application No. PCT/CA2024/051688, filed on Dec. 18, 2024.
(60) Provisional application No. 63/617,741, filed on Jan. 4, 2024.

(51) Int. Cl. A61K 31/65; A61K 9/00; A61K 9/06; A61K 31/167; A61K 31/192; A61K 31/546; A61K 47/10; A61K 47/18; A61K 47/24; A61K 47/26; A61K 47/36; A61K 47/44

(52) U.S. Cl. CPC A61K 31/65; A61K 9/0014; A61K 9/06; A61K 31/167; A61K 31/192; A61K 31/546; A61K 47/10; A61K 47/18; A61K 47/183; A61K 47/24; A61K 47/26; A61K 47/36; A61K 47/44

(57) ABSTRACT

The topical composition includes an amount of an antibiotic and a carrier that has skin penetration enhancement properties. The composition can include about 5 wt % Lidocaine, about 5 wt % Ketoprofen and about 5 wt % Doxycycline. The composition is readily formed as a consumer package. The composition has efficacy as a bite treatment and in particular bacteria conveyed via a tick bite.

### RELATED APPLICATIONS

This application is a continuation-in-part (“bypass”) of PCT Application Serial Number PCT/CA2024/051688 filed 18 Dec. 2024; that in turn claims priority benefit of U.S. Provisional Application Ser. No. 63/617,741 filed 4 Jan. 2024; the contents of which are hereby incorporated by reference.

### FIELD OF THE INVENTION

The invention relates to the field of bite treatment.

### BACKGROUND

Tick bites are commonplace globally and in and of themselves are usually relatively harmless. However, ticks are well known carriers of a bacteria known as Borrelia burgdorferi, a bacterium that can rapidly enter the human circulatory system after a tick bite. Within 3 to 30 days, without identification and proper treatment, infection with B. burgdorferi can lead to a characteristic early exposure bulls-eye rash, fever, chills, headache, fatigue, and muscle and joint aches. Failure to treat the infection can lead to facial palsy, arthritis of large joints such as the hips and knees, irregular heartbeat and inflammation of the brain and spinal cord. Failure to treat this infection can also lead to Lyme Disease.

Infection with B. burgdorferi is generally susceptible to a class of antibiotics known as Tetracyclines. One antibiotic in particular from this family is commonly used to treat infection with B. burgdordferi, namely, oral doxycycline. Cephalosporins have also shown efficacy against tick borne diseases, most specifically, the third generation cephalosporin known as ceftriaxone.

Timely drug administration may provide protection against the manifestations of the infection and may reduce chances of developing Lyme Disease. [Some health authorities are of the opinion that early diagnosis and proper antibiotic treatment of B. burgdorferi is important and can help prevent late Lyme disease. Some health authorities are of the opinion that even a single dose of doxycycline after a tick bite may lower the risk of Lyme disease.

However, in North America and some other jurisdictions these antibiotics are only available by prescription. Furthermore, while doxycycline is available orally, ceftriaxone is only available intravenously. This is problematic in that obtaining a proper healthcare assessment, diagnosis and subsequent prescription/treatment, all promptly after identification of a tick bite, can be difficult, especially in remote settings where bites are most likely.

Oral Tetracyclines and intravenous [IV] Cephalosporins have some risk. Without limitation:
serious side effects of some oral Tetracyclines and IV Cephalosporins include anaphylactic allergic reaction, severe stomach pain, diarrhea, chest pain and irregular heartbeat, plus risk for upsetting natural GI flora balance and risk for development of antibiotic resistance
in pregnant females, tetracyclines are contraindicated due to risk of adverse affects on tooth and bone development in the unborn baby
oral doxycycline can diminish the efficacy of the birth control pill and can cross into the breast milk of a lactating female, thereby putting the baby at risk
in children, because of risk to teeth and bones, oral or intravenous doxycycline is only indicated for severe life-threatening infections that are not treatable by other antibiotics.

### SUMMARY OF THE INVENTION

Forming one aspect of the invention is a consumer package in the form of a topical composition that includes an amount of an antibiotic and a carrier that has skin penetration enhancement properties.

According to another aspect, the antibiotic can be a Tetracycline and/or third generation Cephalosporin.

According to another aspect of the invention, the Tetracycline can be Doxycycline and the Cephalosporin can be Ceftriaxone.

According to another aspect of the invention, the composition can contain other drugs that have anesthetic, an anti-inflammatory and/or analgesic properties.

According to another aspect, the composition can contain Lidocaine and Ketoprofen to provide said anesthetic, anti-inflammatory and analgesic properties.

According to another aspect, the amount of Doxycycline can be 0.1 to 20 total weight percent.

According to another aspect, the amount of Ketoprofen can be 0.1 to 20 total weight percent.

According to another aspect, the amount of Lidocaine can be 0.1 to 10 total weight percent.

According to another aspect, the topical can further comprise at least one of an adjuvant and a non-antibiotic anti-bacterial.

According to another aspect, the carrier can comprise one or more of a diluent, a humectant, a thickener, an emulsifier and a preservative.

According to another aspect, the carrier can comprise one or more of water, glycerin, Xanthan Gum, Disodium EDTA, fractionated coconut oil, isopropyl myristate, Promulgen-D, Polawax, Cetyl Alcohol, Hydroxylated Lecithin, Phenoxyethanol and Liquid Germall Plus.

According to another aspect, the composition can be a cream.

Advantages, features and characteristics of the present invention will become apparent to persons of ordinary skill in the art upon review of the following detailed description with reference to the appended drawings, the latter being briefly described hereinbelow.

### BRIEF DESCRIPTION OF THE DRAWINGS

FIG. 1 is a graph showing the results of a permeation study;

FIG. 2 is a dilution protocol; and

FIG. 3 is a calibration curve generated from the study.

### DETAILED DESCRIPTION

An example embodiment of the invention is a topical composition having the following active ingredients, by weight, in a Permeabase™ carrier: 5% Lidocaine; 5% Ketoprofen; and 5% Doxycycline.

The ingredients of Permeabase™ cream are shown in Table 1

TABLE 1

| n | CAS | Component | Range | Conc. % | Qnty |
|---|---|---|---|---|---|
| 1 | 7732-18-5 | Water | 1 | 30-100 | 67.6 |
| 2 | 8043-29-6 | Glycerin | 3 | 3-10 | 5 |
| 3 | 11138-66-2 | Xanthan Gum | 6 | 0.1-0.3 | 0.1 |
| 4 | 6381-92-6 | Disodium EDTA | 6 | 0.1-0.3 | 0.1 |
| 5 | 73398-61-5 | Fractionated | 3 | 3-10 | 5 |
| | | coconut oil | | | |
| 6 | 142-91-6 | Isopropyl | 3 | 3-10 | 5 |
| | | myristate | | | |
| 7 | 67762-27-0 | Cetostearyl | 3 | 3-10 | 2 |
| | | alcohol | | | |
| | 68439-49-6 | Polyoxyethylene | | | |
| | | (150) | | | |
| | | Monostearate | | | |
| | | (combination is | | | |
| | | Promulgen-D) | | | |
| 8 | 67762-27-0 | Cetostearyl | 3 | 3-10 | 10 |
| | | alcohol | | | |
| | 9004-99-3 | PEG-ISO | | | |
| | | Stearate | | | |
| | 9005-67-8 | Polysorbate 60 | | | |
| | 9005-00-9 | Steareth-20 | | | |
| | | (collectively | | | |
| | | Polawax) | | | |
| 9 | 36653-82-4 | Cetyl Alcohol | 4 | 1-3 | 2 |
| 10 | 8029-76-3 | Hydroxylated | 6 | 0.1-0.3 | 2 |
| | | Lecithin | | | |
| 11 | 122-99-6 | Phenoxyethanol | 5 | 0.3-1.0 | 0.1 |
| 12 | 57-55-6 | Propylene | 5 | 0.3-1.0 | 0.1 |
| | | Glycol | | | |
| | 78491-02-8 | Diazolidinyl | | | |
| | | urea | | | |
| | 55406-53-6 | Iodopropynyl | | | |
| | | Butyl | | | |
| | | Iodopropynyl | | | |
| | | Butyl | | | |
| | | (collectively | | | |
| | | Liquid Germall | | | |
| | | Plus) | | | |
| | | TOTAL: | | | 100 |

The cream is prepared in accordance with Table 2

TABLE 2

| Step | Details |
|---|---|
| A | Slowly blend together #1, 2, 3, & 4 in a stainless steel jacketed, |
| | Heat to 70° C. using a lightnin mixer. |
| B | Mix together #5, 6, 7, 8, 9 & 10 in a separate stainless steel vessel. |
| | Heat to 70° C. |
| C | Slowly combine phase #8 to phase #A while continuing to mix |
| | using lightning mixer. Begin colling process. |
| D | When the temperature of phase #C reaches 50° C., add #11 and |
| | then follow with #12. Continue to mix, cool to 40° C. |
| E | Continue to cool blended batch to 35° C. |

Whereas a specific embodiment is described, variations are possible. Without limitation in this regard:
another carrier with skin penetration enhancement qualities could be substituted for Permeabase
another Tetracycline and/or third generation Cephalosporin could be substituted for Doxycycline
other drugs with anesthetic, anti-inflammatory and/or analgesic properties can be used to replace Lidocaine and Ketoprofen, or omitted altogether
the amount of Doxycycline can be 0.1 to 20 total weight percent of the composition
the amount of Ketoprofen can be 0.1 to 20 total weight percent of the composition
the amount of Lidocaine can be 0.5 to 20 total weight percent of the composition
the carrier can comprise at least one of petrolatum, paraffin, coconut oil, lanolin, beeswax, or C2-C4 alcohol
the skin penetration enhancer can comprise at least one of propylene glycol, benzyl alcohol, and 2-(2-ethoxyethoxy) ethanol
the topical can further comprise aan adjuvant and and a non-antibiotic anti-bacterial
the composition need not be provided as a cream
the composition need not be sold over the counter but could be sold by prescription
whereas use with tick bites is specifically mentioned, it is contemplated that the composition could be used with other wounds in which bacterial infection poses risk, such as bug bites. In this regard, it will be understood that in this document, “bug” should be understood to mean any bug-like creature, including but not limited to ticks, arachnids, insects, mosquitos and mites.

### EXPERIMENTAL

A permeation study using a Franz Diffusion Cell was conducted as follows:
a. Equipment: Teledyne Hanson Phoenix DB-6 dry heat diffusion system
b. Membrane: Strat-M
c. Receptor media: Phosphate Buffer Saline pH 7.4 (PBS)
d. Temperature: 31.5° C. to 32.5° C.
e. Agitation: 400 rpm

Six samples were prepared as per Table 3.

TABLE 3

| Ingredients | Blank | | 0.5 wt % | | 2 wt % | | 5% | | 10% | | 20% | |
|---|---|---|---|---|---|---|---|---|---|---|---|---|
| Doxycycline | 0 | | .125 | g | .50 | g | 1.25 | g | 2.50 | g | 5.00 | g |
| Ethoxy | 1.25 | g | 1.25 | g | 1.25 | g | 1.25 | g | 1.25 | g | 1.25 | g |
| Diglycol | | | | | | | | | | | | |
| Ethyl | 0.5 | g | 0.5 | g | 0.5 | g | 0.5 | g | 0.5 | g | 0.5 | g |
| alcohol | | | | | | | | | | | | |
| Permeabase | 23.25 | g | 23.13 | g | 22.75 | g | 22.00 | g | 20.75 | g | 18.25 | g |

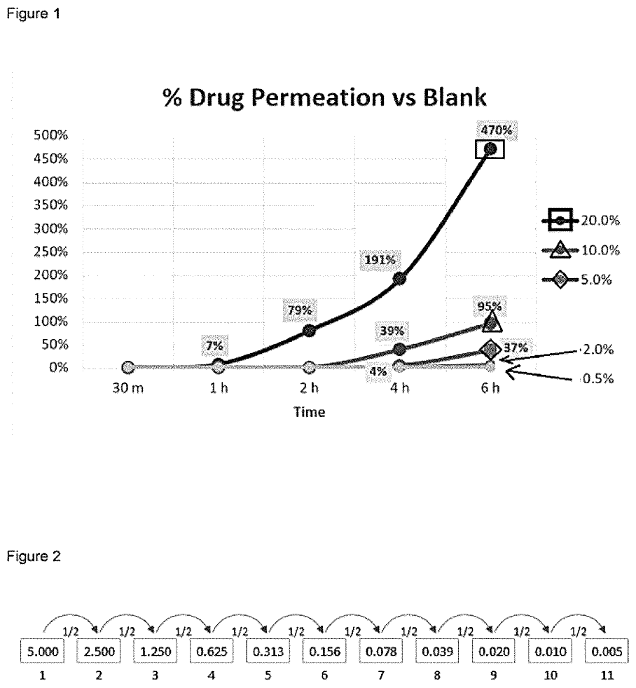

Sampling was done at 30, 60, 120, 240, 480 minutes. The results of the test are shown in FIG. 1.

### Calibration

0.050 grams of Doxycycline was diluted in 10 ml of phosphate buffer and 10 serial ½ dilutions were made mixing ml of the sample with 5 ml of phosphate buffer, as shown in FIG. 2.

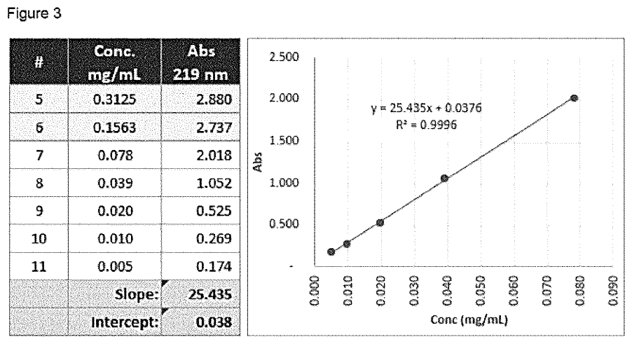

Samples numbered 5 to 11 were measured spectrophotometrically in the range of 200 to 300 nm with the peak being found at 219 nm. A calibration curve, shown in FIG. 3, was constructed using the points 7-11 as 5 and 6 were out of range.

The samples collected were plotted against the calibration curve and their concentration was calculated as shown in Table 4

TABLE 4
Sample Concentration (μg/ml)

| Sample | 30 min | 60 min | 120 min | 240 min | 480 min |
|---|---|---|---|---|---|
| 0.5% | | | | | |
| 2.0% | | | | | 0.33 |
| 5.0% | | | | 0.1 | 7.96 |
| 10.0% | | | | 6.11 | 22.98 |
| 20.0% | | 0.10 | 9.88 | 35.52 | 119.57 |

All samples reached the MIC and MBC of B. burgdorferi [0.25 μg/ml and 16 μg/ml, respectively] at some point, with the most concentrated doing so more quickly.

The lab studies conclude that, via use of the cream, tissue absorption levels of doxycycline can reach the minimal inhibitory concentration (MIC) needed to stop proliferation of B. burgdorferi within 2 hours from the time of application.

Bactericidal levels can be reached in the local tissue within 8 hours of cream application at a 10% doxycycline concentration. The ceftriaxone has an even lower MIC than doxycycline.

From the above, it is reasonable to predict that the consumer package will have significant utility:
the base itself would be expected to provide pain relief and anti-inflammatory properties, thereby rendering it useful as an immediate treatment for tick and other bug and animal bite borne infections
the package—which puts the Doxycycline and/or Ceftriaxone at the origin of the infection—permits the total dose to be relatively low, thereby greatly reducing the risk of serious side effects and rendering the product suitable for treatment of local infection with susceptible bacteria such as B. burgdorferi in the manner of topical anti-biotics

Further, as these antibiotics have also shown treatment efficacy against other bacteria transmitted by bug or animal bites, it is reasonable to predict that the consumer package may have utility as an immediate treatment and/or the prevention of systemic illness.

1. A topical composition that includes an amount of an antibiotic and a carrier that has skin penetration enhancement properties.

2. The topical composition according to claim 1, wherein the antibiotic is a Tetracycline and/or a third generation Cephalosporin.

3. The topical composition according to claim 2, wherein the Tetracycline is Doxycycline and the Cephalosporin is Ceftriaxone.

4. The topical composition according to claim 3, wherein the composition contains other drugs that have anesthetic, anti-inflammatory and/or analgesic properties.

5. The topical composition according to claim 4, wherein the composition contains Lidocaine and Ketoprofen.

6. The topical composition according to claim 5, wherein the composition has about 5 wt % Lidocaine, about 5 wt % Ketoprofen and about 5 wt % Doxycycline.

7. The topical composition according to claim 3, wherein the amount of Doxycycline is 0.1 to 20 total weight percent.

8. The topical composition according to claim 5, wherein the amount of Ketoprofen is 0.1 to 20 total weight percent.

9. The topical composition according to claim 5, wherein the amount of Lidocaine is 0.1 to 10 total weight percent.

10. The topical composition according to claim 1, further comprising at least one of an adjuvant and a non-antibiotic anti-bacterial.

11. The topical composition according to claim 1, wherein the carrier comprises a diluent, a humectant, a thickener, an emulsifier and a preservative.

12. The topical composition according to claim 1, wherein the carrier comprises one or more of water, glycerin, Xanthan Gum, Disodium EDTA, fractionated coconut oil, isopropryl myristate, Promulgen-D, Polawax, Cetyl Alcohol, Hydroxylated Lecithin, Phenoxyethanol and Liquid Germall Plus.

13. Use of the composition of claim 1 for treatment of tick bites.
